(19) United States
(12) Patent Application Publication
(10) Pub. No.: US 2005/0256438 A1
(43) Pub. Date: Nov. 17, 2005

(54) Venipuncture bandage

(76) Inventor: Roberta Lombardozzi, Chestnut Hill, MA (US)

Correspondence Address: Patrick D. Archibald;Lambert & Associates, 92 State Street, Boston, MA, 02109, US

(21) Appl. No.: 10/880,077

(22) Filed: Jun. 29, 2004

Related U.S. Application Data
(60) Provisional application No. 60/570,148, filed on May 13, 2004.

(51) Int. Cl. A61F 13/00

(52) U.S. Cl. 602/53

(57) ABSTRACT

Provided is a pressure bandage suitable for achieving complete hemostasis at a venipuncture site. The bandage includes an adhesive backing, upon which a non-compressible, absorbent pad is centrally affixed. The backing is inelastic, which along with the non-compressible pad, provides a non-resilient dressing capable of applying the pressure necessary to achieve hemostasis. The pad typically comprises a plurality of plies of dense cotton gauze. The pressure bandage may be applied by one individual, quickly and efficiently, at a low cost.

### CROSS-REFERENCE TO RELATED APPLICATIONS

This application is based on and claims priority from provisional patent application Ser. No. 60/570,148 filed on May 13, 2004.

### BACKGROUND OF THE INVENTION

1. Field of the Invention

This invention relates generally to the field of bandages and more particularly to a specialized pressure bandage.

2. Description of the Related Art

In a variety of health care settings including hospitals, laboratories, clinics and medical offices, venipuncture (perforating a vein with a needle) is an everyday practice. The most common reasons for perforation of a vein include blood drawing, insertion of a venous catheter for the administration of intravenous drugs, or to keep an intravenous line open in the event a drug needs to be administered quickly to a patient.

After a venipuncture and subsequent to the removal of the needle or catheter from a vein, sufficient localized pressure must be applied to the venipuncture site in order to promote rapid hemostasis (clotting of blood). This localized pressure is accomplished by the application of a pressure dressing directly to the venipuncture site.

In current practice, the most common technique is for a nurse or technician to make his/her own pressure dressing. This is accomplished by taking a sterile gauze out of package, folding it in order to provide the amount of bulk necessary for compression, and placing the gauze over the venipucture site. The practitioner then tears an elongated piece of tape from a roll, attaches it over the gauze and adheres the elongated strips to the skin of the patient. Depending on the technique used by the practitioner, the patient may be required to hold the gauze in place while the practitioner tears the tape from the roll. This technique is inefficient in that it requires time-consuming assembly of the component parts of the dressing, and often the assistance of the patient.

In addition to inefficient practices, prior art techniques often incorporate some ineffective dressing components, such as the use of a resilient pad. The softness of a resilient pad lacks the tautness, or firmness, necessary to apply the proper amount of pressure to the venipuncture site. Such prior art techniques can also utilize elastic adhesive strips that can stretch. The stretchability of such strips causes them to be inadequate to provide the necessary pressure required for venous compression and rapid clotting. Other techniques involve pads that are too thin, or bandages that require complex construction.

For example, the prior art patent granted to Hansen, U.S. Pat. No. 4,377,159 discloses a pressure bandage that includes a compressible, resilient material such as resilient foam. These types of bandages are undesirable as noted above. Additionally, the prior art patent granted to Simpson, U.S. Pat. No. 4,971,046 discloses a surgical pressure plaster that includes a pad comprising a resilient pressure pad attached to the tacky side of tape that is highly elastic, and undesirable for the above-mentioned reasons.

Thus, in order to maximize the effectiveness of the use of a pressure dressing at a venipuncture site, the following criteria must be met:

1. The dressing must be sterile.

2. The dressing must be simply constructed so that it requires only one person to remove the needle or catheter with one hand, and apply the dressing with the other hand.

3. The dressing must be constructed as to apply sufficient pressure to the vein until complete hemostasis is achieved, which usually occurs in 30 minutes.

4. There must be speed of application in securing the dressing.

5. The pad of the dressing must be absorbent.

6. The bandage must be cost-effective.

7. The dressing must be easy to remove.

### BRIEF SUMMARY OF THE INVENTION

Accordingly, it is an object of the present invention to provide a pressure bandage that is sterile and is so simply constructed that it can be applied by a single person.

It is a further object of the present invention to be able to apply sufficient pressure so as to achieve complete hemostasis.

It is also an object of the present invention to provide a pressure bandage that may be applied quickly, is easy to remove, and provides absorbency.

It is yet a further object of the present invention to provide a pressure bandage that is inexpensive to manufacture, thereby achieving cost-effectiveness.

It is another object of the present invention to provide a pressure bandage that is not so soft or resilient as to compromise the pressure necessary to provide hemostasis.

It is a further object of this invention to provide a backing that covers the pad, such backing composed of a non-elastic adhesive tape capable of providing the necessary tautness to maintain compression on the vinipuncture site.

The present invention is a pressure bandage that combines the use of a non-resilient cotton or cotton-like gauze pad and a non-resilient, non-stretchable adhesive tape with easily removable protective strips to provide effective hemostasis and ease of application.

### BRIEF DESCRIPTION OF THE SEVERAL VIEWS OF THE DRAWINGS

These and other features, aspects and advantages of the present invention will become better understood with reference to the following description, appended claims, and accompanying drawings where:

FIG. 1 is a perspective view of the pressure bandage of the present invention.

FIG. 2 is a an elevated side view of the pressure bandage of the present invention with its removal strip tabs overlapping the pad.

FIG. 3 is a perspective view of the pressure bandage of this invention in place on a patient's arm.

### DETAILED DESCRIPTION OF THE INVENTION

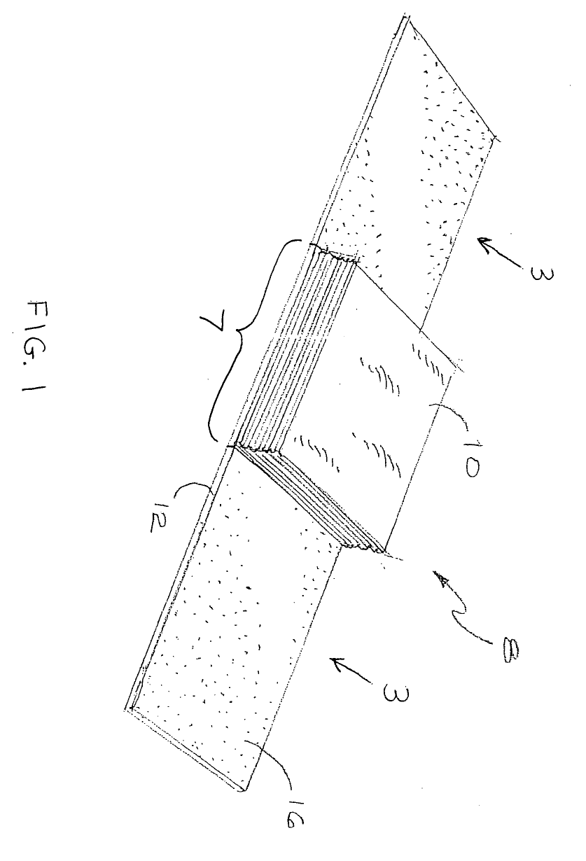

FIG. 1 illustrates a perspective view of pressure bandage 8 of the present invention showing pad 10, which can be made of readily available dense cotton gauze or equivalent material. In a preferred embodiment the thickness of the pad should be 16 ply, which is approximately ¼ inch thick, the length of the pad should be approximately 1½ inches long, and the width of the pad should be approximately 1 inch. The cotton gauze should be of a density that is non-compressible. The backing 12 must be non-stretchable and non-resilient. Such backing can be made of readily available non-resilient adhesive tape. Examples of such tape include Transpore or Micropore tape from 3M. Backing 12 preferably includes an anterior side 1, which includes a fastening section 3. Fastening section 3 includes an adhesive portion 16, and a pad retention section 7. Backing 12 also includes a posterior side 2, which is non-adhesive in the preferred embodiment. Although fastening section 3 includes adhesive portion 16 in the preferred embodiment, it should be clear to those skilled in the art that the securing of the pressure bandage 8 to a patient 6 could be achieved by other suitable means. One example, which is not intended as limiting, could include a backing 12 that wraps around the arm of a patient 6 and is secured to itself by means of velcro, tackiness, or other equivalent methods. Additionally, the backing 12 could comprise a variety of shapes and sizes including, but not limited to rectangular strips, squares, circles, butterfly shapes and others. Backing 12 could also comprise various embodiments as known in the art, such as perforations, transparency, opacity, designs, drawings, text and other items.

Pad 10 could mirror the shape and size of backing 12, or could be a consistent shape, such as a square. Further, pad 10 could include additives as are well-known in the art, such as medications, anti-bacterial treatments and the like. Such additives may be applied to the outer surface of pad 10, impregnated within the plies, or any combination thereof.

In a preferred embodiment backing 12 can be 5 inches in length and 1 inch in width. Pad 10 is centered on the 5 inch length of backing 12. The preferred dimensions of pad 10 and backing 12 provide for sufficient hemostasis, and the pressure bandage 8 is of a size that is comfortable for the patient to wear.

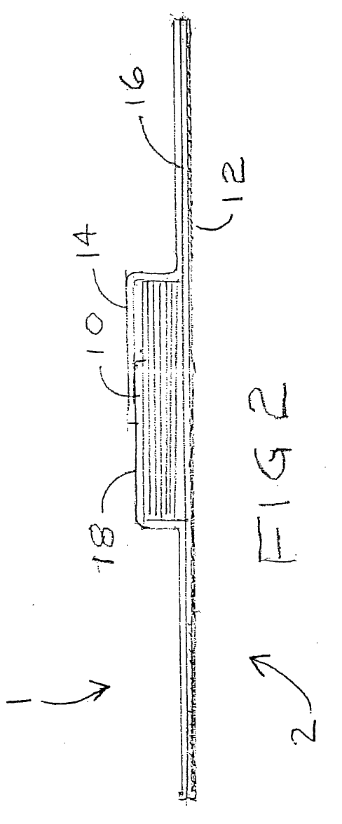

As seen in FIG. 2 a pair of removable protective strip tabs being first and second strip tabs 14 and 18 cover adhesive portion 16 of backing 12. First and second strip tabs 14 and 18 have a surface well known in the art designed to removably adhere to the adhesive portions 16 of backing 12. First strip tab 14 in a preferred embodiment can measure 2½ inches in length from one end to the center of the pressure bandage 8, and 1 inch in width. Second strip tab 18 can measure approximately 2¾ inches in length and 1 inch in width, which allows for a ¼ inch strip tab overlap at the center of backing 12, over the pad 10. This overlap provides for easy grasping and removal of strip tabs 14 and 18 in the preferred embodiment.

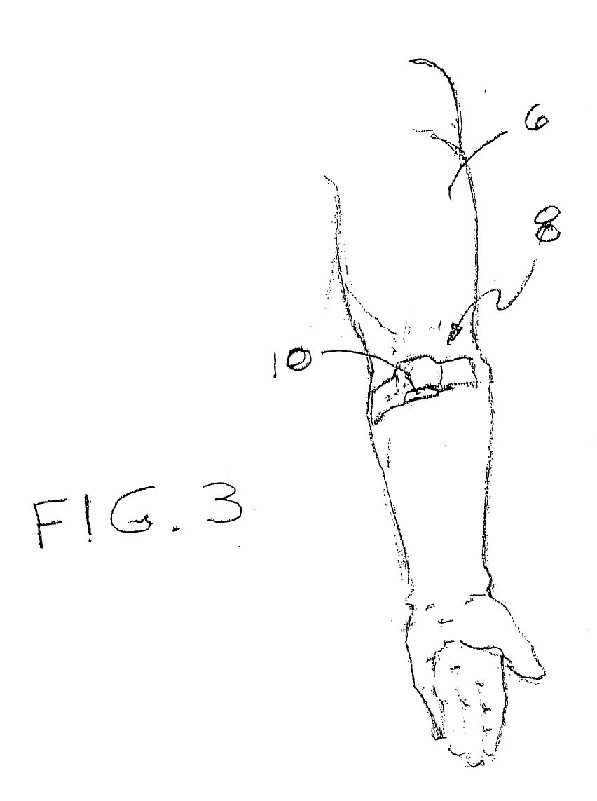

FIG. 3 illustrates pressure bandage 8 of the present invention in place on the arm of a patient 6. Pressure bandage 8 of the present invention has a thick, non-compressible pad 10 and is of a larger overall size compared to ordinary prior art bandages. Pressure bandage 8 of the present invention has a non-elastic adhesive tape backing which provides the compression necessary for hemostasis at a venipuncture site. It will be clear to those skilled in the art that the pressure bandage 8 of the present invention will be equally useful in any situation where it is desirable to apply pressure to a certain location on a patient.

In the preferred method of using the venipuncture bandage of the invention, the practitioner pulls back on first and second strip tabs 14 and 18 sufficiently to reveal pad 10. With a first hand the practitioner places the pad 10 over the venipuncture site. With the second hand the practitioner removes the needle or catheter while applying pressure on pad 10 with the first hand. Once the needle or catheter has been removed, and while pressure is being applied with the first hand over the pad 10, the second hand is free to discard the needle or catheter. That same hand can then remove both of the protective strip tabs 14 and 18 from the pressure bandage 8 and attach the adhesive portion 16 of backing 12 firmly to the skin of the patient at the venipuncture site.

To maintain sterility, the venipuncture pressure bandage of this invention can be packaged individually or can be placed in a multiunit sterile dispenser that is constructed to dispense one, or more, sterile venipuncture pressure bandages at a time.

Although the present invention has been described with reference to particular embodiments, it will be apparent to those skilled in the art that variations and modifications can be substituted therefore without departing from the principles and spirit of the invention.

1-5. (canceled)

6. A pressure bandage comprising:
a backing, said backing included a backing length and a backing width, and wherein said backing includes an anterior side and a posterior side, and wherein said anterior side of said backing includes a fastening section, and wherein said anterior side includes a pad retention section; wherein said pad retention section includes a pad retention section width measuring substantially equivalent to said backing width, and wherein said pad retention section includes a pad retention section length, wherein said pad retention section length is less than said backing length, and wherein pad retention section is located substantially in the center of said backing;
a pad affixed to said pad retention section, wherein said pad resides substantially within said pad retention section, and wherein said pad is substantially non-compressible.

7. The pressure bandage of claim 6, wherein said fastening section comprises an adhesive portion, said adhesive portion being located substantially on said anterior side of said backing.

8. The pressure bandage of claim 7, wherein said pad comprises at least one layer of gauze.

9. The pressure bandage of claim 8, wherein said pad comprises sixteen layers of gauze.

10. The pressure bandage of claim 9, wherein said backing is substantially non-stretchable.

11. The pressure bandage of claim 10, wherein said backing is substantially non-resilient.

12. The pressure bandage of claim 11, wherein said backing length measures approximately five inches.

13. The pressure bandage of claim 12, wherein said pad retention section length measures approximately one and one half inches.

14. The pressure bandage of claim 6, further comprising at least one strip tab, said strip tab being removably attached to a fastening section of said adhesive portion of said backing.

15. The pressure bandage of claim 14, further comprising a first strip tab and a second strip tab, said first strip tab and said second strip tab being removably attached to a fastening section of said adhesive portion of said backing.

16. The pressure bandage of claim 15, wherein a sum of said first strip tab length and said second strip tab length measures approximately equivalent to said backing length.

17. The pressure bandage of claim 16, wherein a sum of said first strip tab length and said second strip tab length measures greater than said backing length.

18. The pressure bandage of claim 17, wherein said first strip tab and said second strip tab are positioned on said anterior side of said backing so as to create an overlapping portion.

19. The pressure bandage of claim 18, wherein said overlapping portion lies substantially over said pad.

20. The pressure bandage of claim 19, wherein said pad comprises at least one layer of gauze.

21. The pressure bandage of claim 20, wherein said pad comprises sixteen layers of gauze.

22. The pressure bandage of claim 21, wherein said backing is substantially non-stretchable.

23. The pressure bandage of claim 22, wherein said backing is substantially non-resilient.

24. The pressure bandage of claim 23, wherein said backing length measures approximately five inches.

25. The pressure bandage of claim 24, wherein said pad retention section length measures approximately one and one half inches.

26. A method for applying a pressure bandage, said method comprising the steps of:
(a) pulling back on a first strip tab, and pulling back on a second strip tab from an adhesive tape;
(b) revealing a pad;
(c) placing said pad over a desired site with a first hand;
(d) withdrawing an instrument from a subject with a second hand, while applying pressure on said pad with said first hand;
(e) discarding said instrument with said second hand;
(f) removing said first strip tab and removing said second strip tab with said second hand; and
(g) attaching said adhesive tape to said subject with said second hand.

27. The method of claim 26, wherein said adhesive tape is substantially non-stretchable, and said pad is substantially non-resilient.

28. The method of claim 27, wherein said pad is substantially non-compressible.
